(19) United States
(12) Patent Application Publication
(10) Pub. No.: US 2019/0300473 A1
(43) Pub. Date: Oct. 3, 2019

(54) COMPOSITION COMPRISING 1-CHLORO-2,2-DIFLUOROETHANE

(71) Applicant: Arkema France, Colombes (FR)

(72) Inventors: Dominique GARRAIT, Charly (FR); Charlotte HERDT, Poissy (FR); Camille SCHERPEREEL, Bourgoin Jallieu (FR)

(73) Assignee: Arkema France, Colombes (FR)

(21) Appl. No.: 16/336,305

(22) PCT Filed: Sep. 25, 2017
(86) PCT No.: PCT/FR2017/052558

§ 371 Date: Mar. 25, 2019

(30) Foreign Application Priority Data
Sep. 27, 2016 (FR) 1659073
Sep. 27, 2016 (FR) 1659074
Sep. 27, 2016 (FR) 1659075
Sep. 27, 2016 (FR) 1659076
Sep. 27, 2016 (FR) 1659077
Sep. 27, 2016 (FR) 1659078

(51) Int. Cl. C07C 211/15; C07C 19/12; C07C 21/18

(52) U.S. Cl. CPC C07C 211/15; C07C 21/18; C07C 19/12

(57) ABSTRACT

Compositions including 1-chloro-2,2-difluoroethane, and the uses thereof. A composition including 1-chloro-2,2-difluoroethane and at least one additional compound selected from 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; the composition including at least 30% by weight of 1-chloro-2,2-difluoroethane, based on the total weight of the composition.

### TECHNICAL FIELD OF THE INVENTION

The present invention relates to a composition comprising 1-chloro-2,2-difluoroethane. The present invention also relates to the use of this composition as synthesis intermediate or starting products or as foam blowing agent.

### Technological Background of the Invention

1-Chloro-2,2-difluoroethane is a known foam blowing agent and may also be used as a starting product in the preparation of products intended for agrochemistry or for the field of pharmaceuticals.

Ecological, toxicological and environmental constraints require the use of compositions in which the contents of certain impurities are reduced.

### SUMMARY OF THE INVENTION

The present invention relates to a composition comprising 1-chloro-2,2-difluoroethane and at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; said composition comprising at least 30% by weight of 1-chloro-2,2-difluoroethane, based on the total weight of the composition.

Preferably, the present invention relates to a composition comprising 1-chloro-2,2-difluoroethane and at least one additional compound selected from the group consisting of 1,1-difluoroethylene, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; said composition comprising at least 30% by weight of 1-chloro-2,2-difluoroethane, based on the total weight of the composition.

Preferably, said composition comprises at least 40% by weight of 1-chloro-2,2-difluoroethane, based on the total weight of the composition, advantageously at least 50% by weight of 1-chloro-2,2-difluoroethane, preferably at least 60% by weight of 1-chloro-2,2-difluoroethane, in particular at least 80% by weight of 1-chloro-2,2-difluoroethane, more particularly at least 90% by weight of 1-chloro-2,2-difluoroethane, favorably at least 95% by weight of 1-chloro-2,2-difluoroethane.

According to a preferred embodiment, said composition comprises less than 5% by weight of said at least one additional compound, based on the total weight of the composition, advantageously less than 4% by weight of said at least one additional compound, preferably less than 3% by weight of said at least one additional compound, more preferentially less than 2% by weight of said at least one additional compound, in particular less than 1% by weight of said at least one additional compound, more particularly less than 1000 ppm of said at least one additional compound, based on the total weight of the composition.

According to a preferred embodiment, said composition also comprises one of the compounds selected from the group consisting of trans-1,2-dichloroethylene, 1,1,2-trifluoroethane and 1,2-dichloro-1-fluoroethane.

According to a preferred embodiment, said composition comprises 1-chloro-2,2-difluoroethane, 1,1-dichloroethylene and optionally or not at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl.

According to a preferred embodiment, said composition comprises 1-chloro-2,2-difluoroethane, 1,1-dichloroethylene, trans-1,2-dichloroethylene and optionally at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1-chloro-2,2-difluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl.

According to a preferred embodiment, said composition comprises 1-chloro-2,2-difluoroethane, 1,1-dichloroethylene, trans-1,2-dichloroethylene, 1-chloro-2,2,2-trifluoroethane and 1,1,2-trifluoroethane.

Preferably, said composition comprises at least 99.5% by weight of 1-chloro-2,2-difluoroethane, less than 50 ppm of 1,1,2-trifluoroethane, less than 100 ppm of 1-chloro-2,2,2-trifluoroethane and less than 5000 ppm of trans-1,2-dichloroethylene and less than 50 ppm of 1,1-dichloroethylene.

In particular, said composition also comprises less than 500 ppm of H₂O, advantageously less than 400 ppm of H₂O, preferably less than 300 ppm of H₂O, in particular less than 100 ppm of H₂O, more particularly less than 10 ppm.

More particularly, said composition also comprises at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-1,2,2-trifluoroethane, cis-1-chloro-2-fluoroethylene, 2,2-dichloro-1,1,1-trifluoroethane, 1,1-dichloro-1-fluoroethane, 1-chloro-2,2-difluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, and 1-fluoroethylene.

According to a second aspect of the present invention, said composition is used as starting product for the synthesis of 2,2-difluoroethylamine.

According to a third aspect of the present invention, a process for preparing a compound of formula CF₂CH₂NHCH₂A (I) is provided. Said process comprises the steps of:

(a) providing a composition as claimed in any one of claims 1 to 11;

(b) reaction between the composition from step (a) and an amine of formula ACH₂NH₂ (II) in order to obtain the compound of formula CF₂CH₂NHCH₂A (I) in which A is selected from the group consisting of a pyrid-2-yl, pyrid-4-yl or pyrid-3-yl radical, optionally substituted in position 6 by a fluorine, chlorine or bromine atom, or a methyl, trifluoromethyl or trifluoromethoxy radical, or is a pyridazin-3-yl radical optionally substituted in position 6 by a chlorine atom or a methyl radical, or is a pyrazin-3-yl, 2-chloropyrazin-5-yl or 1,3-thiazol-5-yl radical optionally substituted in position 2 by a chlorine atom or a methyl radical, or is a pyrimidinyl, pyrazolyl, thiophenyl, oxazolyl, isoxazolyl, 1,2,4-oxadiazolyl, isothiazolyl, 1,2,4-triazolyl or 1,2,5-thiadiazolyl radical optionally substituted by a fluorine, chlorine or bromine atom or a cyano or nitro group or C₁-C₄-alkyl optionally substituted by F and/or Cl, C₁-C₃-alkylthio optionally substituted by F and/or Cl, C₁-C₃-alkylsulfonyl optionally substituted by F and/or Cl, or is a pyrid-3-yl radical of formula

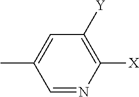

in which X is F, Cl, Br, I, C₁-C₆ alkyl, C₁-C₆-haloalkyl; and Y is F, Cl, Br, I, C₁-C₆ alkyl, C₁-C₆-haloalkyl, C₁-C₆-haloalkoxy, —N₃, —CN.

According to another aspect of the present invention, said composition is used as reagents for the synthesis of vinylidene fluoride and/or 1,2-difluoroethylene.

According to another aspect of the present invention, said composition is used as foam blowing agent or as refrigerant.

According to another aspect of the present invention, said composition is used as reagent or synthesis intermediate for the preparation of 2,2-difluoroethanol or for the preparation of compounds comprising a sulfone, ether, ester or methacrylate functional group or for the preparation of ethyl difluoro bromoacetate.

### DETAILED DESCRIPTION OF THE INVENTION

The present invention relates to a composition comprising 1-chloro-2,2-difluoroethane. Said composition also comprises at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl.

Preferably, the composition comprises 1-chloro-2,2-difluoroethane and at least one additional compound selected from the group consisting of 1,1-difluoroethylene, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; said composition comprising at least 30% by weight of 1-chloro-2,2-difluoroethane, based on the total weight of the composition.

The term “at least one” refers to one or more, and includes compositions comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 additional compound(s) as described in the present application. When there are several additional compounds, the content expressed corresponds to the content of each of said additional compounds, independent of the other additional compounds that may be present in the composition.

In addition, said composition comprises at least 30% by weight of 1-chloro-2,2-difluoroethane based on the total weight of the composition.

Preferably, the composition comprises at least 40% by weight of 1-chloro-2,2-difluoroethane, based on the total weight of the composition, advantageously at least 50% by weight of 1-chloro-2,2-difluoroethane, preferably at least 60% by weight of 1-chloro-2,2-difluoroethane, in particular at least 80% by weight of 1-chloro-2,2-difluoroethane, more particularly at least 90% by weight of 1-chloro-2,2-difluoroethane, favorably at least 95% by weight of 1-chloro-2,2-difluoroethane.

Said composition comprises less than 40% by weight of said at least one additional compound, based on the total weight of the composition, advantageously less than 35% by weight, preferably less than 30% by weight, more preferentially less than 25% by weight, in particular less than 20% by weight, more particularly less than 10% by weight of said at least one additional compound, based on the total weight of the composition.

Preferably, the composition comprises less than 5% by weight of said at least one additional compound, based on the total weight of the composition, advantageously less than 4% by weight of said at least one additional compound, preferably less than 3% by weight of said at least one additional compound, more preferentially less than 2% by weight of said at least one additional compound, in particular less than 1% by weight of said at least one additional compound, more particularly less than 1000 ppm of said at least one additional compound, based on the total weight of the composition.

According to a particular embodiment, said at least one additional compound may also be selected from trans-1,2-dichloroethylene, 1,1,2-trifluoroethane and 1,2-dichloro-1-fluoroethane.

In any one of the compositions described in the present application below, the content of each of the different compounds therein is detailed below.

Preferably, the content of 1,1-dichloroethylene in said composition may be less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm. Alternatively, said composition may also be devoid of 1,1-dichloroethylene, that is to say contain less than 1 ppm based on the total weight of the composition.

The content of 1,1-difluoroethylene in said composition may be less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm. Alternatively, said composition may also be devoid of 1,1-difluoroethylene, that is to say contain less than 1 ppm based on the total weight of the composition.

The content of 1,1,1-trifluoroethane in said composition may be less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm. Alternatively, said composition may also be devoid of 1,1,1-trifluoroethane, that is to say contain less than 1 ppm based on the total weight of the composition.

The content of chlorotrifluoroethylene in said composition may be less than 1000 ppm, advantageously less than 500 ppm, preferably less than 250 ppm, more preferentially less than 100 ppm, in particular less than 50 ppm. Alternatively, said composition may also be devoid of chlorotrifluoroethylene, that is to say contain less than 1 ppm based on the total weight of the composition.

The content of 1-chloro-1-fluoroethylene in said composition may be less than 1% by weight based on the total weight of the composition, advantageously less than 5000 ppm, preferably less than 2500 ppm, more preferentially less than 1000 ppm. Alternatively, said composition may also be devoid of 1-chloro-1-fluoroethylene, that is to say contain less than 1 ppm based on the total weight of the composition.

The content of 1,1-difluoroethane in said composition may be less than 1% by weight based on the total weight of the composition, advantageously less than 5000 ppm, preferably less than 2500 ppm, more preferentially less than 1000 ppm, in particular less than 500 ppm, more particularly less than 300 ppm. Alternatively, said composition may also be devoid of 1,1-difluoroethane, that is to say contain less than 1 ppm based on the total weight of the composition.

The content of trans-1-chloro-2-fluoroethylene in said composition may be less than 2% by weight based on the total weight of the composition, advantageously less than 1% by weight, preferably less than 8000 ppm, more preferentially less than 5000 ppm, in particular less than 4000 ppm, more particularly less than 3000 ppm. Alternatively, said composition may also be devoid of trans-1-chloro-2-fluoroethylene, that is to say contain less than 1 ppm based on the total weight of the composition.

The content of 1,1,2-trifluoroethane in said composition may be less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm.

The content of 1-chloro-2,2,2-trifluoroethane in said composition may be less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm.

The content of 1-chloro-1,2,2-trifluoroethane in said composition may be less than 1% by weight based on the total weight of the composition, advantageously less than 5000 ppm, preferably less than 4000 ppm, more preferentially less than 3000 ppm, in particular less than 2000 ppm, more particularly less than 1600 ppm. Alternatively, said composition may also be devoid of 1-chloro-1,2,2-trifluoroethane, that is to say contain less than 1 ppm based on the total weight of the composition.

The content of cis-1-chloro-2-fluoroethylene in said composition may be less than 5% by weight based on the total weight of the composition, advantageously less than 4% by weight, preferably less than 3% by weight, more preferentially less than 2% by weight, in particular less than 1% by weight, more particularly less than 8000 ppm. Alternatively, said composition may also be devoid of cis-1-chloro-2-fluoroethylene, that is to say contain less than 1 ppm based on the total weight of the composition.

The content of 2,2-dichloro-1,1,1-trifluoroethane in said composition may be less than 1% by weight based on the total weight of the composition, advantageously less than 5000 ppm, preferably less than 4000 ppm, more preferentially less than 3000 ppm, in particular less than 2000 ppm, more particularly less than 1000 ppm. Alternatively, said composition may also be devoid of 2,2-dichloro-1,1,1-trifluoroethane, that is to say contain less than 1 ppm based on the total weight of the composition.

The content of 1,1-dichloro-1-fluoroethane in said composition may be less than 1% by weight based on the total weight of the composition, advantageously less than 5000 ppm, preferably less than 4000 ppm, more preferentially less than 3000 ppm, in particular less than 2000 ppm, more particularly less than 1000 ppm. Alternatively, said composition may also be devoid of 1,1-dichloro-1-fluoroethane, that is to say contain less than 1 ppm based on the total weight of the composition.

The content of 1,2-dichloro-1-fluoroethylene in said composition may be less than 1% by weight based on the total weight of the composition, advantageously less than 5000 ppm, preferably less than 4000 ppm, more preferentially less than 3000 ppm, in particular less than 2000 ppm, more particularly less than 1000 ppm. Alternatively, said composition may also be devoid of 1,2-dichloro-1-fluoroethylene, that is to say contain less than 1 ppm based on the total weight of the composition.

The content of 1,1-dichloro-2-fluoroethylene in said composition may be less than 1% by weight based on the total weight of the composition, advantageously less than 5000 ppm, preferably less than 4000 ppm, more preferentially less than 3000 ppm, in particular less than 2000 ppm, more particularly less than 1000 ppm. Alternatively, said composition may also be devoid of 1,1-dichloro-2-fluoroethylene, that is to say contain less than 1 ppm based on the total weight of the composition.

The content of 1,2-dichloro-1,1-difluoroethane in said composition may be less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm. Alternatively, said composition may also be devoid of 1,2-dichloro-1,1-difluoroethane, that is to say contain less than 1 ppm based on the total weight of the composition.

The content of trans-1,2-dichloroethylene in said composition may be less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm.

The content of 1,1-dichloro-2,2-difluoroethane in said composition may be less than 5% by weight based on the total weight of the composition, advantageously less than 4% by weight, preferably less than 3% by weight, more preferentially less than 2% by weight, in particular less than 1% by weight. Alternatively, said composition may also be devoid of 1,1-dichloro-2,2-difluoroethane, that is to say contain less than 1 ppm based on the total weight of the composition.

The content of cis-1,2-dichloroethylene in said composition may be less than 40% by weight based on the total weight of the composition, advantageously less than 30% by weight, preferably less than 20% by weight, more preferentially less than 15% by weight, in particular less than 10% by weight, more particularly less than 5% by weight. Alternatively, said composition may also be devoid of cis-1,2-dichloroethylene, that is to say contain less than 1 ppm based on the total weight of the composition.

The content of 1,2,2-trichloro-1,1-difluoroethane in said composition may be less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm. Alternatively, said composition may also be devoid of 1,2,2-trichloro-1,1-difluoroethane, that is to say contain less than 1 ppm based on the total weight of the composition.

The content of 1,2-dichloro-1-fluoroethane in said composition may be less than 10% by weight based on the total weight of said composition, advantageously less than 9% by weight, preferably less than 8% by weight, more preferentially less than 7% by weight, in particular less than 6% by weight, more particularly less than 5% by weight, favorably less than 4% by weight. Alternatively, said composition may also be devoid of 1,2,2-trichloro-1,1-difluoroethane, that is to say contain less than 1 ppm based on the total weight of the composition.

The content of 1,1,1-trichloroethane in said composition may be less than 1% by weight based on the total weight of the composition, advantageously less than 5000 ppm, preferably less than 4000 ppm, more preferentially less than 3000 ppm, in particular less than 2000 ppm, more particularly less than 1000 ppm. Alternatively, said composition may also be devoid of 1,1,1-trichloroethane, that is to say contain less than 1 ppm based on the total weight of the composition.

The content of 1,1-dichloroethane in said composition may be less than 1% by weight based on the total weight of the composition, advantageously less than 5000 ppm, preferably less than 4000 ppm, more preferentially less than 3000 ppm, in particular less than 2000 ppm, more particularly less than 1000 ppm. Alternatively, said composition may also be devoid of 1,1-dichloroethane, that is to say contain less than 1 ppm based on the total weight of the composition.

The content of 1,1,2-trichloro-1-fluoroethane in said composition may be less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm. Alternatively, said composition may also be devoid of 1,1,2-trichloro-1-fluoroethane, that is to say contain less than 1 ppm based on the total weight of the composition.

The content of trichloroethylene in said composition may be less than 3% by weight based on the total weight of the composition, advantageously less than 2% by weight, preferably less than 10000 ppm, more preferentially less than 7500 ppm, in particular less than 5000 ppm, more particularly less than 3500 ppm. Alternatively, said composition may also be devoid of trichloroethylene, that is to say contain less than 1 ppm based on the total weight of the composition.

The content of 1,1,2-trichloro-2-fluoroethane in said composition may be less than 5% by weight based on the total weight of the composition, advantageously less than 4% by weight, preferably less than 3% by weight, more preferentially less than 2% by weight, in particular less than 1% by weight. Alternatively, said composition may also be devoid of 1,1,2-trichloro-2-fluoroethane, that is to say contain less than 1 ppm based on the total weight of the composition.

The content of 1,1,2-trichloroethane in said composition may be less than 5% by weight based on the total weight of the composition, advantageously less than 4% by weight, preferably less than 3% by weight, more preferentially less than 2% by weight, in particular less than 1% by weight. Alternatively, said composition may also be devoid of 1,1,2-trichloroethane, that is to say contain less than 1 ppm based on the total weight of the composition.

The content of 1,1,2,2-tetrachloro-1-fluoroethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm. Alternatively, said composition may also be devoid of 1,1,2,2-tetrachloro-1-fluoroethane, that is to say contain less than 1 ppm based on the total weight of the composition.

The content of tetrachloroethylene in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm. Alternatively, said composition may also be devoid of tetrachloroethylene, that is to say contain less than 1 ppm based on the total weight of the composition.

The content of 1,1,1,2-tetrachloroethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm. Alternatively, said composition may also be devoid of 1,1,1,2-tetrachloroethane, that is to say contain less than 1 ppm based on the total weight of the composition.

The content of 1,1,2,2-tetrachloroethane in said composition may be less than 5% by weight based on the total weight of the composition, advantageously less than 4% by weight, preferably less than 3% by weight, more preferentially less than 2% by weight, in particular less than 1% by weight. Alternatively, said composition may also be devoid of 1,1,1,2-tetrachloroethane, that is to say contain less than 1 ppm based on the total weight of the composition.

The content of 1,1,1,2,2-pentachloroethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1,1,1,2,2-pentachloroethane, that is to say contain less than 1 ppm based on the total weight of the composition.

The content of tetrachloromethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of tetrachloromethane, that is to say contain less than 1 ppm based on the total weight of the composition.

The content of 1,2,2-trichloro-1-fluoroethylene in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1,2,2-trichloro-1-fluoroethylene, that is to say contain less than 1 ppm based on the total weight of the composition.

The content of isomers of chloropropene in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of isomers of chloropropene, that is to say contain less than 1 ppm based on the total weight of the composition.

The content of 1,2-dichloro-1-fluoroethylene in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1,2-dichloro-1-fluoroethylene, that is to say contain less than 1 ppm based on the total weight of the composition.

The content of 1-chloro-2-fluoroethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1-chloro-2-fluoroethane, that is to say contain less than 1 ppm based on the total weight of the composition.

The content of 1-chloro-1-fluoroethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1-chloro-1-fluoroethane, that is to say contain less than 1 ppm based on the total weight of the composition.

The content of 1,1-chloro-2,2,2-trifluoroethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1,1-chloro-2,2,2-trifluoroethane, that is to say contain less than 1 ppm based on the total weight of the composition.

The content of 1,1,1-trichloro-2,2-difluoroethane in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1,1,1-trichloro-2,2-difluoroethane, that is to say contain less than 1 ppm based on the total weight of the composition.

The content of 1-chloroethylene in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1-chloroethylene, that is to say contain less than 1 ppm based on the total weight of the composition.

The content of 1-fluoroethylene in said composition may be less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm. Alternatively, said composition may also be devoid of 1-fluoroethylene, that is to say contain less than 1 ppm based on the total weight of the composition.

The content of H₂O in said composition may be less than 1% by weight based on the total weight of the composition, advantageously less than 5000 ppm, preferably less than 3000 ppm, more preferentially less than 1000 ppm, in particular less than 750 ppm, more particularly less than 500 ppm, favorably less than 250 ppm, more favorably less than 100 ppm, even more favorably less than 50 ppm, most favorably less than 10 ppm.

The content of HCl in said composition may be such that the pH thereof is between 6 and 9, advantageously between 6.5 and 8.5, preferably between 6.5 and 8.0, more preferentially between 7.0 and 8.0. The content of HCl may be less than 5%, advantageously less than 2%, preferably less than 5000 ppm, more preferentially less than 1000 ppm, in particular less than 100 ppm, more particularly less than 1 ppm.

According to a first aspect of the invention, said composition comprises 1-chloro-2,2-difluoroethane and 1,1-dichloroethylene. Preferably, said composition comprises 1-chloro-2,2-difluoroethane, 1,1-dichloroethylene and optionally at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O. Said composition may also comprise HCl.

Said composition may thus comprise at least 40% by weight, advantageously at least 50% by weight, preferably at least 60% by weight, in particular at least 80% by weight, more particularly at least 90% by weight, favorably at least 95% by weight of 1-chloro-2,2-difluoroethane; a content of 1,1-dichloroethylene of less than 2% by weight, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm; and optionally less than 5% by weight, advantageously less than 4% by weight, preferably less than 3% by weight, more preferentially less than 2% by weight, in particular less than 1% by weight, more particularly less than 1000 ppm of at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O; based on the total weight of the composition.

Advantageously, said composition may comprise 1-chloro-2,2-difluoroethane, 1,1-dichloroethylene, trans-1,2-dichloroethylene and optionally at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1-chloro-2,2-difluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O. Preferably, said composition may thus comprise at least 40% by weight, advantageously at least 50% by weight, preferably at least 60% by weight, in particular at least 80% by weight, more particularly at least 90% by weight, favorably at least 95% by weight of 1-chloro-2,2-difluoroethane; a content of 1,1-dichloroethylene of less than 2% by weight, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm; a content of trans-1,2-dichloroethylene of less than 30% by weight, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm; and optionally less than 5% by weight, advantageously less than 4% by weight, preferably less than 3% by weight, more preferentially less than 2% by weight, in particular less than 1% by weight, more particularly less than 1000 ppm of at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O; based on the total weight of the composition.

Preferably, said composition may comprise at least 80% by weight of 1-chloro-2,2-difluoroethane; a content of 1,1-dichloroethylene of less than 50 ppm, a content of trans-1,2-dichloroethylene of less than 20% by weight and less than 500 ppm of H₂O, advantageously less than 400 ppm of H₂O, preferably less than 300 ppm of H₂O, in particular less than 100 ppm of H₂O, more particularly less than 10 ppm of H₂O, based on the total weight of the composition. Said composition may have a pH of between 6.5 and 8.

In particular, said composition may comprise at least 95% by weight of 1-chloro-2,2-difluoroethane; a content of 1,1-dichloroethylene of less than 50 ppm, a content of trans-1,2-dichloroethylene of less than 5% by weight, preferably less than 3% by weight, and less than 500 ppm of H₂O, advantageously less than 400 ppm of H₂O, preferably less than 300 ppm of H₂O, in particular less than 100 ppm of H₂O, more particularly less than 10 ppm of H₂O, based on the total weight of the composition. Said composition may have a pH of between 6.5 and 8.

Preferably, said composition may comprise 1-chloro-2,2-difluoroethane, 1,1-dichloroethylene, trans-1,2-dichloroethylene, 1-chloro-2,2,2-trifluoroethane and 1,1,2-trifluoroethane. Said composition may comprise at least 40% by weight, advantageously at least 50% by weight, preferably at least 60% by weight, in particular at least 80% by weight, more particularly at least 90% by weight, favorably at least 95% by weight of 1-chloro-2,2-difluoroethane; a content of 1,1-dichloroethylene of less than 2% by weight, advantageously less than 1% by weight, preferably less than 0,5% by weight, more preferentially less than 1000 ppm, in particular less than 500 ppm, more particularly less than 100 ppm, favorably less than 50 ppm, more favorably less than 10 ppm, even more favorably less than 1 ppm; a content of trans-1,2-dichloroethylene of less than 30% by weight, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm; a content of 1-chloro-1,2,2-trifluoroethane of less than 1% by weight, advantageously less than 5000 ppm, preferably less than 4000 ppm, more preferentially less than 3000 ppm, in particular less than 2000 ppm, more particularly less than 1600 ppm, and a content of 1,1,2-trifluoroethane of less than 5% by weight, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm, based on the total weight of the composition.

Preferably, said composition may comprise at least 99.5% by weight of 1-chloro-2,2-difluoroethane, less than 50 ppm of 1,1,2-trifluoroethane, less than 100 ppm of 1-chloro-2,2,2-trifluoroethane and less than 5000 ppm of trans-1,2-dichloroethylene and less than 50 ppm of 1,1-dichloroethylene.

Optionally, said composition may comprise at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O. Preferably said composition may comprise H₂O. In particular, said composition may comprise less than 500 ppm of H₂O, advantageously less than 400 ppm of H₂O, preferably less than 300 ppm H₂O, in particular less than 100 ppm of H₂O, more particularly less than 10 ppm of H₂O. Thus, said composition may comprise at least 99.5% by weight of 1-chloro-2,2-difluoroethane, less than 50 ppm of 1,1,2-trifluoroethane, less than 100 ppm of 1-chloro-2,2,2-trifluoroethane and less than 5000 ppm of trans-1,2-dichloroethylene, less than 50 ppm of 1,1-dichloroethylene and less than 500 ppm of H₂O, advantageously less than 400 ppm of H₂O, preferably less than 300 ppm of H₂O, in particular less than 100 ppm of H₂O, more particularly less than 10 ppm of H₂O. The pH of said composition may be between 6.5 and 8.

Preferably, said composition may comprise 1-chloro-2,2-difluoroethane, 1,1-dichloroethylene, trans-1,2-dichloroethylene, 1-chloro-2,2,2-trifluoroethane, 1,1,2-trifluoroethane, and at least one, two, three, four, five, six, seven, eight, nine, ten, eleven, twelve, thirteen, fourteen, fifteen, sixteen, seventeen, eighteen, nineteen, twenty, twenty-one or all of the additional compounds selected from the group consisting of 1,1-difluoroethylene, 1,1,1-trifluoroethane, 1-chloro-1-fluoroethylene, 1,1-difluoroethane, trans-1-chloro-2-fluoroethylene, 1-chloro-1,2,2-trifluoroethane, cis-1-chloro-2-fluoroethylene, 1,2-dichloro-1-fluoroethylene, 1,2-dichloro-1,1-difluoroethane, 1,1-dichloro-2,2-difluoroethane, cis-1,2-dichloroethylene, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,2,2-tetrachloroethane, tetrachloromethane, 1,2,2-trichloro-1-fluoroethylene, isomers of chloropropene and H₂O.

In particular, said composition may comprise:
at least 40% by weight, advantageously at least 50% by weight, preferably at least 60% by weight of 1-chloro-2,2-difluoroethane;
a content of 1,1-dichloroethylene of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm;
a content of trans-1,2-dichloroethylene in said composition may be less than 30% by weight based on the total weight of the composition, advantageously less than 20% by weight, preferably less than 15% by weight, more preferentially less than 10% by weight;
a content of 1-chloro-2,2,2-trifluoroethane in said composition may be less than 5% by weight based on the total weight of the composition, advantageously less than 4% by weight, preferably less than 3% by weight, more preferentially less than 2% by weight, in particular less than 1% by weight;
a content of 1,1,2-trifluoroethane in said composition may be less than 5% by weight based on the total weight of the composition, advantageously less than 4% by weight, preferably less than 3% by weight, more preferentially less than 2% by weight, in particular less than 1% by weight; and
at least one, two, three, four, five, six, seven, eight, nine, ten, eleven, twelve, thirteen, fourteen, fifteen, sixteen, seventeen, eighteen, nineteen, twenty, twenty-one, or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   cis-1,2-dichloroethylene at a content of less than 40% by weight based on the total weight of the composition, advantageously less than 30% by weight, preferably less than 20% by weight, more preferentially less than 15% by weight, in particular less than 10% by weight, more particularly less than 5% by weight,
   1,2-dichloro-1-fluoroethane at a content of less than 10% by weight based on the total weight of said composition, advantageously less than 9% by weight, preferably less than 8% by weight, more preferentially less than 7% by weight, in particular less than 6% by weight, more particularly less than 5% by weight, favorably less than 4% by weight,
   1,1,2-trichloro-1-fluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm,
   trichloroethylene at a content of less than 3% by weight based on the total weight of the composition, advantageously less than 2% by weight, preferably less than 10 000 ppm, more preferentially less than 7500 ppm, in particular less than 5000 ppm, more particularly less than 3500 ppm,
   1,1,2-trichloro-2-fluoroethane at a content of less than 5% by weight based on the total weight of the composition, advantageously less than 4% by weight, preferably less than 3% by weight, more preferentially less than 2% by weight, in particular less than 1% by weight,
   1,1,2-trichloroethane at a content of less than 5% by weight based on the total weight of the composition, advantageously less than 4% by weight, preferably less than 3% by weight, more preferentially less than 2% by weight, in particular less than 1% by weight,
   1,1,2,2-tetrachloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   tetrachloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,2,2-tetrachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   tetrachloromethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,2,2-trichloro-1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   isomers of chloropropene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm; and
   less than 500 ppm, advantageously less than 400 ppm, preferably less than 300 ppm, in particular less than 100 ppm of H₂O, more particularly less than 10 ppm of H₂O;
   based on the total weight of the composition.

As indicated above, the content of HCl in any one of the compositions described above may be such that the pH thereof is between 6 and 9, advantageously between 6.5 and 8.5, preferably between 6.5 and 8.0, more preferentially between 7.0 and 8.0.

According to a second aspect of the present invention, a composition comprising 1-chloro-2,2-difluoroethane and trans-1,2-dichloroethylene is provided.

Preferably, said composition comprises at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10 000 ppm, more favorably less than 5000 ppm, based on the total weight of the composition.

Preferably, said composition comprises 1-chloro-2,2-difluoroethane, trans-1,2-dichloroethylene and at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 1,1,2-trifluoroethane, 1,2-dichloro-1-fluoroethane, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; said composition comprising at least 30% by weight of 1-chloro-2,2-difluoroethane, based on the total weight of the composition.

Preferably, said composition comprises at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10 000 ppm, more favorably less than 5000 ppm; and less than 35% by weight of said at least one additional compound, advantageously less than 30% by weight, preferably less than 10% by weight of said at least one additional compound, more preferentially less than 15% by weight of said at least one additional compound, in particular less than 5% by weight of said at least one additional compound, more particularly less than 1% by weight of said at least one additional compound, based on the total weight of the composition.

In particular, said composition comprises 1-chloro-2,2-difluoroethane, trans-1,2-dichloroethylene, cis-1,2-dichloroethylene and 1-chloro-2,2,2-trifluoroethane.

Preferably, the content by weight of cis-1,2-dichloroethylene is less than 35% based on the total weight of the composition, advantageously less than 30% by weight, preferably less than 20% by weight, more preferentially less than 15% by weight, in particular less than 5% by weight, more particularly less than 1% by weight, favorably less than 4000 ppm.

Preferably, the content by weight of 1-chloro-2,2-difluoroethane is less than 10% based on the total weight of the composition, advantageously less than 5% by weight, preferably less than 1% by weight, more preferentially less than 5000 ppm, in particular less than 2000 ppm.

Thus, said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10 000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm, based on the total weight of the composition.

Preferably, said composition may thus comprise at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl. In particular, said at least one additional compound may be selected from the group consisting of 1,1-difluoroethylene, 1,1,1-trifluoroethane, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,2-dichloro-1-fluoroethylene, 1,2-dichloro-1,1-difluoroethane, 1,1-dichloro-2,2-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,2,2-tetrachloroethane, 1,1,1,2,2-pentachloroethane, tetrachloromethane, 1,2,2-trichloro-1-fluoroethylene, isomers of chloropropene and H₂O.

Thus, said composition may comprise 1-chloro-2,2-difluoroethane, trans-1,2-dichloroethylene, cis-1,2-dichloroethylene, 1-chloro-2,2,2-trifluoroethane and at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl.

Said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm; and less than 5% by weight, advantageously less than 4% by weight, preferably less than 3% by weight, more preferentially less than 2% by weight, in particular less than 1% by weight, more particularly less than 1000 ppm of said at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; based on the total weight of the composition.

According to a particular embodiment, said composition may comprise 1-chloro-2,2-difluoroethane, trans-1,2-dichloroethylene, cis-1,2-dichloroethylene, 1-chloro-2,2,2-trifluoroethane and at least one additional compound selected from the group consisting of 1,1-difluoroethylene, 1,1,1-trifluoroethane, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 1,1-dichloroethylene, 1,2-dichloro-1-fluoroethylene, 1,2-dichloro-1,1-difluoroethane, 1,1-dichloro-2,2-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,2,2-tetrachloroethane, 1,1,1,2,2-pentachloroethane, tetrachloromethane, 1,2,2-trichloro-1-fluoroethylene, isomers of chloropropene and optionally H₂O.

Thus, said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm; and less than 5% by weight, advantageously less than 4% by weight, preferably less than 3% by weight, more preferentially less than 2% by weight, in particular less than 1% by weight, more particularly less than 1000 ppm of said at least one additional compound selected from the group consisting of 1,1-difluoroethylene, 1,1,1-trifluoroethane, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 1,1-dichloroethylene, 1,2-dichloro-1-fluoroethylene, 1,2-dichloro-1,1-difluoroethane, 1,1-dichloro-2,2-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,2,2-tetrachloroethane, 1,1,1,2,2-pentachloroethane, tetrachloromethane, 1,2,2-trichloro-1-fluoroethylene, isomers of chloropropene and optionally H₂O. More preferentially, said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm; and less than 5% by weight, advantageously less than 4% by weight, preferably less than 3% by weight, more preferentially less than 2% by weight, in particular less than 1% by weight, more particularly less than 1000 ppm of said at least one, two, three, four, five, six, seven, eight, nine, ten, eleven, twelve, thirteen, fourteen, fifteen, sixteen, seventeen, eighteen, nineteen, twenty, twenty-one, twenty-two or all of the additional compound(s) selected from the group consisting of 1,1-difluoroethylene, 1,1,1-trifluoroethane, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 1,1-dichloroethylene, 1,2-dichloro-1-fluoroethylene, 1,2-dichloro-1,1-difluoroethane, 1,1-dichloro-2,2-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,2,2-tetrachloroethane, 1,1,1,2,2-pentachloroethane, tetrachloromethane, 1,2,2-trichloro-1-fluoroethylene, isomers of chloropropene and optionally H₂O.

Thus, said composition may comprise:
at least 40% by weight of 1-chloro-2,2-difluoroethane, advantageously at least 50% by weight, preferably at least 60% by weight;
a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10 000 ppm, more favorably less than 5000 ppm;
a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm;
a content by weight of 1-chloro-2,2-difluoroethane of less than 10%, advantageously less than 5% by weight, preferably less than 1% by weight, more preferentially less than 5000 ppm, in particular less than 2000 ppm;
at least one, two, three, four, five, six, seven, eight, nine, ten, eleven, twelve, thirteen, fourteen, fifteen, sixteen, seventeen, eighteen, nineteen, twenty, twenty-one, twenty-two or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm,
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   1,2-dichloro-1-fluoroethane at a content of less than 10% by weight based on the total weight of said composition, advantageously less than 9% by weight, preferably less than 8% by weight, more preferentially less than 7% by weight, in particular less than 6% by weight, more particularly less than 5% by weight, favorably less than 4% by weight,
   1,1,2-trichloro-1-fluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm,
   trichloroethylene at a content of less than 3% by weight based on the total weight of the composition, advantageously less than 2% by weight, preferably less than 10 000 ppm, more preferentially less than 7500 ppm, in particular less than 5000 ppm, more particularly less than 3500 ppm,
   1,1,2-trichloro-2-fluoroethane at a content of less than 5% by weight based on the total weight of the composition, advantageously less than 4% by weight, preferably less than 3% by weight, more preferentially less than 2% by weight, in particular less than 1% by weight,
   1,1,2-trichloroethane at a content of less than 5% by weight based on the total weight of the composition, advantageously less than 4% by weight, preferably less than 3% by weight, more preferentially less than 2% by weight, in particular less than 1% by weight,
   1,1,2,2-tetrachloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   tetrachloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,2,2-tetrachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,1,2,2-pentachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   tetrachloromethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,2,2-trichloro-1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   isomers of chloropropene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm; and
   optionally less than 500 ppm, advantageously less than 400 ppm, preferably less than 300 ppm, in particular less than 100 ppm of H₂O, more particularly less than 10 ppm of H₂O;
   based on the total weight of the composition.

According to another particular embodiment, said composition may comprise 1-chloro-2,2-difluoroethane, trans-1,2-dichloroethylene, cis-1,2-dichloroethylene, 1-chloro-2,2,2-trifluoroethane and at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene and 1,1,2,2-tetrachloroethane. Advantageously, said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm; and less than 5% by weight, advantageously less than 4% by weight, preferably less than 3% by weight, more preferentially less than 2% by weight, in particular less than 1% by weight, more particularly less than 1000 ppm of said at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene and 1,1,2,2-tetrachloroethane. Preferably, said composition may comprise 1-chloro-2,2-difluoroethane, trans-1,2-dichloroethylene, cis-1,2-dichloroethylene, 1-chloro-2,2,2-trifluoroethane and at least one, two, three, four, five, six, seven, eight, nine, ten, eleven, twelve, thirteen, fourteen, fifteen, sixteen, seventeen, eighteen, nineteen, twenty, twenty-one or all of the additional compounds selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene and 1,1,2,2-tetrachloroethane. More preferentially, said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm; and less than 5% by weight, advantageously less than 4% by weight, preferably less than 3% by weight, more preferentially less than 2% by weight, in particular less than 1% by weight, more particularly less than 1000 ppm of said at least one, two, three, four, five, six, seven, eight, nine, ten, eleven, twelve, thirteen, fourteen, fifteen, sixteen, seventeen, eighteen, nineteen, twenty, twenty-one or all of the additional compounds selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-1,2,2-trifluoroethane, cis-1-chloro-2-fluoroethylene, 2,2-dichloro-1,1,1-trifluoroethane, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene and 1,1,2,2-tetrachloroethane.

Thus, said composition may comprise:
at least 40% by weight of 1-chloro-2,2-difluoroethane, advantageously at least 50% by weight, preferably at least 60% by weight;
a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10 000 ppm, more favorably less than 5000 ppm;
a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm;
a content by weight of 1-chloro-2,2-difluoroethane of less than 10%, advantageously less than 5% by weight, preferably less than 1% by weight, more preferentially less than 5000 ppm, in particular less than 2000 ppm, and
at least one, two, three, four, five, six, seven, eight, nine, ten, eleven, twelve, thirteen, fourteen, fifteen, sixteen, seventeen, eighteen, nineteen, twenty, twenty-one, or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   chlorotrifluoroethylene at a content of less than 1000 ppm, advantageously less than 500 ppm, preferably less than 250 ppm, more preferentially less than 100 ppm, in particular less than 50 ppm;
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm.
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   2,2-dichloro-1,1,1-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   1,2,2-trichloro-1,1-difluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm.
   1,2-dichloro-1-fluoroethane at a content of less than 10% by weight based on the total weight of said composition, advantageously less than 9% by weight, preferably less than 8% by weight, more preferentially less than 7% by weight, in particular less than 6% by weight, more particularly less than 5% by weight, favorably less than 4% by weight,
   1,1,2-trichloro-1-fluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm,
   trichloroethylene at a content of less than 3% by weight based on the total weight of the composition, advantageously less than 2% by weight, preferably less than 10 000 ppm, more preferentially less than 7500 ppm, in particular less than 5000 ppm, more particularly less than 3500 ppm,
   1,1,2-trichloro-2-fluoroethane at a content of less than 5% by weight based on the total weight of the composition, advantageously less than 4% by weight, preferably less than 3% by weight, more preferentially less than 2% by weight, in particular less than 1% by weight,
   1,1,2-trichloroethane at a content of less than 5% by weight based on the total weight of the composition, advantageously less than 4% by weight, preferably less than 3% by weight, more preferentially less than 2% by weight, in particular less than 1% by weight,
   1,1,2,2-tetrachloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   tetrachloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,2,2-tetrachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, based on the total weight of the composition.

According to another particular embodiment, said composition may comprise 1-chloro-2,2-difluoroethane, trans-1,2-dichloroethylene, cis-1,2-dichloroethylene, 1-chloro-2,2,2-trifluoroethane and at least one compound selected from the group consisting of 1,1-difluoroethylene, 1-chloro-1-fluoroethylene, trans-1-chloro-2-fluoroethylene, 1,1,2-trifluoroethane, 1-chloro-1,2,2-trifluoroethane, cis-1-chloro-2-fluoroethylene, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2,2-pentachloroethane and H₂O. Advantageously, said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm; and less than 5% by weight, advantageously less than 4% by weight, preferably less than 3% by weight, more preferentially less than 2% by weight, in particular less than 1% by weight, more particularly less than 1000 ppm of said at least one additional compound selected from the group consisting of 1,1-difluoroethylene, 1-chloro-1-fluoroethylene, trans-1-chloro-2-fluoroethylene, 1,1,2-trifluoroethane, 1-chloro-1,2,2-trifluoroethane, cis-1-chloro-2-fluoroethylene, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2,2-pentachloroethane and H₂O. Preferably, said composition may comprise 1-chloro-2,2-difluoroethane, trans-1,2-dichloroethylene, cis-1,2-dichloroethylene, 1-chloro-2,2,2-trifluoroethane and at least one, two, three, four, five, six, seven, eight, nine, ten, eleven, twelve, thirteen, fourteen, fifteen, sixteen, seventeen, eighteen, or all of the additional compounds selected from the group consisting of 1,1-difluoroethylene, 1-chloro-1-fluoroethylene, trans-1-chloro-2-fluoroethylene, 1,1,2-trifluoroethane, 1-chloro-1,2,2-trifluoroethane, cis-1-chloro-2-fluoroethylene, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2,2-pentachloroethane and H₂O. More preferentially, said composition may comprise at least 40% by weight of 1-chloro-2,2-difluoroethane, advantageously at least 50% by weight, preferably at least 60% by weight; and a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10000 ppm, more favorably less than 5000 ppm; a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm; and a content by weight of 1-chloro-2,2-difluoroethane of less than 10%, advantageously less than 5% by weight, preferably less than 1% by weight, more preferentially less than 5000 ppm, in particular less than 2000 ppm; and less than 5% by weight, advantageously less than 4% by weight, preferably less than 3% by weight, more preferentially less than 2% by weight, in particular less than 1% by weight, more particularly less than 1000 ppm of said at least one, two, three, four, five, six, seven, eight, nine, ten, eleven, twelve, thirteen, fourteen, fifteen, sixteen, seventeen, eighteen or all of the additional compounds selected from the group consisting of 1,1-difluoroethylene, 1-chloro-1-fluoroethylene, trans-1-chloro-2-fluoroethylene, 1,1,2-trifluoroethane, 1-chloro-1,2,2-trifluoroethane, cis-1-chloro-2-fluoroethylene, 1,1-dichloroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2,2-pentachloroethane and H₂O.

Thus, said composition may comprise:
at least 40% by weight of 1-chloro-2,2-difluoroethane, advantageously at least 50% by weight, preferably at least 60% by weight;
a content by weight of trans-1,2-dichloroethylene of less than 25%, advantageously less than 20% by weight, preferably less than 15% by weight, more preferentially less than 10% by weight, in particular less than 5% by weight, more particularly less than 3% by weight, favorably less than 10 000 ppm, more favorably less than 5000 ppm;
a content by weight of cis-1,2-dichloroethylene of less than 35%, advantageously less than 30% by weight, preferably less than 20% by weight, more preferentially less than 15% by weight, in particular less than 5% by weight, more particularly less than 1% by weight, favorably less than 4000 ppm;
a content by weight of 1-chloro-2,2-difluoroethane of less than 10%, advantageously less than 5% by weight, preferably less than 1% by weight, more preferentially less than 5000 ppm, in particular less than 2000 ppm, and
at least one, two, three, four, five, six, seven, eight, nine, ten, eleven, twelve, thirteen, fourteen, fifteen, sixteen, seventeen, eighteen, nineteen, twenty, twenty-one, or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1-chloro-1-fluoroethylene at a content of less than 1% by weight based on the total weight of the composition, advantageously less than 5000 ppm, preferably less than 2500 ppm, more preferentially less than 1000 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm.
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   1,2,2-trichloro-1,1-difluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm.
   1,2-dichloro-1-fluoroethane at a content of less than 10% by weight based on the total weight of said composition, advantageously less than 9% by weight, preferably less than 8% by weight, more preferentially less than 7% by weight, in particular less than 6% by weight, more particularly less than 5% by weight, favorably less than 4% by weight,
   1,1,2-trichloro-1-fluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm,
   trichloroethylene at a content of less than 3% by weight based on the total weight of the composition, advantageously less than 2% by weight, preferably less than 10 000 ppm, more preferentially less than 7500 ppm, in particular less than 5000 ppm, more particularly less than 3500 ppm,
   1,1,2-trichloro-2-fluoroethane at a content of less than 5% by weight based on the total weight of the composition, advantageously less than 4% by weight, preferably less than 3% by weight, more preferentially less than 2% by weight, in particular less than 1% by weight,
   1,1,2-trichloroethane at a content of less than 5% by weight based on the total weight of the composition, advantageously less than 4% by weight, preferably less than 3% by weight, more preferentially less than 2% by weight, in particular less than 1% by weight,
   1,1,2,2-tetrachloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   tetrachloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,1,2,2-pentachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   H₂O at a content of less than 1% by weight based on the total weight of the composition, advantageously less than 5000 ppm, preferably less than 3000 ppm, more preferentially less than 1000 ppm, in particular less than 750 ppm, more particularly less than 500 ppm, favorably less than 250 ppm, more favorably less than 100 ppm, even more favorably less than 50 ppm, most favorably less than 10 ppm, based on the total weight of the composition.

According to another aspect of the present invention, a composition comprising 1-chloro-2,2-difluoroethane and H₂O is provided. Said composition may comprise at least 40% by weight of 1-chloro-2,2-difluoroethane, based on the total weight of the composition, advantageously at least 50% by weight of 1-chloro-2,2-difluoroethane, preferably at least 60% by weight of 1-chloro-2,2-difluoroethane, in particular at least 80% by weight of 1-chloro-2,2-difluoroethane, more particularly at least 90% by weight of 1-chloro-2,2-difluoroethane, favorably at least 95% by weight of 1-chloro-2,2-difluoroethane.

According to a preferred embodiment, the content of H₂O in the composition is less than 2000 ppm, advantageously less than 1000 ppm, preferably less than 750 ppm, more preferentially less than 550 ppm, in particular less than 100 ppm, more particularly less than 10 ppm of H₂O.

Thus, said composition may comprise at least 40% by weight, advantageously at least 50% by weight, preferably at least 60% by weight, in particular at least 80% by weight, more particularly at least 90% by weight, favorably at least 95% by weight of 1-chloro-2,2-difluoroethane based on the total weight of the composition; and a content of H₂O of less than 2000 ppm, advantageously less than 1000 ppm, preferably less than 750 ppm, more preferentially less than 550 ppm, in particular less than 100 ppm, more particularly less than 10 ppm of H₂O.

Said composition may comprise at least 80% by weight of 1-chloro-2,2-difluoroethane based on the total weight of the composition; and a content of H₂O of less than 750 ppm.

In addition, said composition may comprise at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1-chloro-2,2-difluoroethane, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1,1-dichloroethylene and 1-fluoroethylene. Preferably, said composition comprises less than 40% by weight of said at least one additional compound, based on the total weight of the composition, advantageously less than 35% by weight, preferably less than 30% by weight, more preferentially less than 25% by weight, in particular less than 20% by weight, more particularly less than 10% by weight of said at least one additional compound, based on the total weight of the composition. In particular, said composition comprises less than 5% by weight of said at least one additional compound, based on the total weight of the composition, advantageously less than 4% by weight of said at least one additional compound, preferably less than 3% by weight of said at least one additional compound, more preferentially less than 2% by weight of said at least one additional compound, in particular less than 1% by weight of said at least one additional compound, more particularly less than 1000 ppm of said at least one additional compound, based on the total weight of the composition.

Advantageously, said composition may comprise 1-chloro-2,2-difluoroethane, H₂O, trans-1,2-dichloroethylene and 1,1-dichloroethylene. Said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane,
a content of H₂O of less than 2000 ppm, advantageously less than 1000 ppm, preferably less than 750 ppm, more preferentially less than 550 ppm, in particular less than 100 ppm H₂O, more particularly less than 10 ppm of H₂O,
a content of trans-1,2-dichloroethylene of less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm,
a content of 1,1-dichloroethylene of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm.

Said composition may also comprise cis-1,2-dichloroethylene at a content of less than 40% by weight based on the total weight of the composition, advantageously less than 30% by weight, preferably less than 20% by weight, more preferentially less than 15% by weight, in particular less than 10% by weight, more particularly less than 5% by weight.

Said composition may also comprise 1,1,2-trifluoroethane at a content of less than 5% by weight, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm; and/or 1-chloro-2,2,2-trifluoroethane at a content of less than 5% by weight, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm based on the total weight of the composition.

Said composition may also comprise at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1,1-dichloroethylene and 1-fluoroethylene; preferably at a content of less than 5% by weight based on the total weight of the composition, advantageously less than 4% by weight, preferably less than 3% by weight, more preferentially less than 2% by weight, in particular less than 1% by weight, more particularly less than 1000 ppm of said at least one additional compound based on the total weight of the composition.

Advantageously, said additional compound may be selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-1,2,2-trifluoroethane, cis-1-chloro-2-fluoroethylene, 2,2-dichloro-1,1,1-trifluoroethane, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,2,2-tetrachloroethane, 1,1,1,2,2-pentachloroethane; preferably at a content of less than 5% by weight based on the total weight of the composition, advantageously less than 4% by weight, preferably less than 3% by weight, more preferentially less than 2% by weight, in particular less than 1% by weight, more particularly less than 1000 ppm of said at least one additional compound based on the total weight of the composition. Thus, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane,
H₂O at a content of less than 2000 ppm, advantageously less than 1000 ppm, preferably less than 750 ppm, more preferentially less than 550 ppm, in particular less than 100 ppm H₂O, more particularly less than 10 ppm of H₂O,
trans-1,2-dichloroethylene at a content of less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm,
1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
cis-1,2-dichloroethylene at a content of less than 40% by weight based on the total weight of the composition, advantageously less than 30% by weight, preferably less than 20% by weight, more preferentially less than 15% by weight, in particular less than 10% by weight, more particularly less than 5% by weight,
1,1,2-trifluoroethane at a content of less than 5% by weight, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm; and/or 1-chloro-2,2,2-trifluoroethane at a content of less than 5% by weight, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm, and
at least one, two, three, four, five, six, seven, eight, nine, ten, eleven, twelve, thirteen, fourteen, fifteen, sixteen, seventeen, eighteen, nineteen, twenty, twenty-one, or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   chlorotrifluoroethylene at a content of less than 1000 ppm, advantageously less than 500 ppm, preferably less than 250 ppm, more preferentially less than 100 ppm, in particular less than 50 ppm;
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   2,2-dichloro-1,1,1-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,1,2-trichloro-1-fluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm,
   trichloroethylene at a content of less than 3% by weight based on the total weight of the composition, advantageously less than 2% by weight, preferably less than 10 000 ppm, more preferentially less than 7500 ppm, in particular less than 5000 ppm, more particularly less than 3500 ppm,
   1,1,2-trichloroethane at a content of less than 5% by weight based on the total weight of the composition, advantageously less than 4% by weight, preferably less than 3% by weight, more preferentially less than 2% by weight, in particular less than 1% by weight,
   1,1,2,2-tetrachloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,2,2-tetrachloroethane at a content of less than 5% by weight based on the total weight of the composition, advantageously less than 4% by weight, preferably less than 3% by weight, more preferentially less than 2% by weight, in particular less than 1% by weight,
   1,1,1,2,2-pentachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm, based on the total weight of the composition.

In particular, said additional compound may be selected from the group consisting of 1,1-difluoroethylene, 1-chloro-1-fluoroethylene, trans-1-chloro-2-fluoroethylene, 1-chloro-1,2,2-trifluoroethane, cis-1-chloro-2-fluoroethylene, 1,2-dichloro-1,1-difluoroethane, 1,1-dichloro-2,2-difluoroethane, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,2,2-tetrachloroethane, 1,1,1,2,2-pentachloroethane; preferably at a content of less than 5% by weight based on the total weight of the composition, advantageously less than 4% by weight, preferably less than 3% by weight, more preferentially less than 2% by weight, in particular less than 1% by weight, more particularly less than 1000 ppm of said at least one additional compound based on the total weight of the composition. Thus, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane,
H₂O at a content of less than 2000 ppm, advantageously less than 1000 ppm, preferably less than 750 ppm, more preferentially less than 550 ppm, in particular less than 100 ppm H₂O, more particularly less than 10 ppm of H₂O,
trans-1,2-dichloroethylene at a content of less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm,
1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
cis-1,2-dichloroethylene at a content of less than 40% by weight based on the total weight of the composition, advantageously less than 30% by weight, preferably less than 20% by weight, more preferentially less than 15% by weight, in particular less than 10% by weight, more particularly less than 5% by weight,
1,1,2-trifluoroethane at a content of less than 5% by weight, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm; and/or 1-chloro-2,2,2-trifluoroethane at a content of less than 5% by weight, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm, and
at least one, two, three, four, five, six, seven, eight, nine, ten, eleven, twelve, thirteen, fourteen, fifteen, sixteen, or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1-chloro-1-fluoroethylene at a content of less than 1% by weight based on the total weight of the composition, advantageously less than 5000 ppm, preferably less than 2500 ppm, more preferentially less than 1000 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   1,1,2-trichloro-1-fluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm,
   trichloroethylene at a content of less than 3% by weight based on the total weight of the composition, advantageously less than 2% by weight, preferably less than 10 000 ppm, more preferentially less than 7500 ppm, in particular less than 5000 ppm, more particularly less than 3500 ppm,
   1,1,2-trichloroethane at a content of less than 5% by weight based on the total weight of the composition, advantageously less than 4% by weight, preferably less than 3% by weight, more preferentially less than 2% by weight, in particular less than 1% by weight,
   1,1,2,2-tetrachloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,2,2-tetrachloroethane at a content of less than 5% by weight based on the total weight of the composition, advantageously less than 4% by weight, preferably less than 3% by weight, more preferentially less than 2% by weight, in particular less than 1% by weight,
   1,1,1,2,2-pentachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm, based on the total weight of the composition.

According to another preferred embodiment, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane,
H₂O at a content of less than 2000 ppm, advantageously less than 1000 ppm, preferably less than 750 ppm, more preferentially less than 550 ppm, in particular less than 100 ppm H₂O, more particularly less than 10 ppm of H₂O,
at least one, two, three, four, five, six, seven, eight, nine, ten, eleven, twelve, thirteen, or all of the additional compounds selected from the group consisting of: 1,1-difluoroethylene, 1-chloro-1-fluoroethylene, 1,1-difluoroethane, 1,1-dichloroethylene, 1,2-dichloro-1-fluoroethylene, 1,2-dichloro-1,1-difluoroethane, 1,1-dichloro-2,2-difluoroethane, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene and 1-fluoroethylene.

Preferably, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane,
H₂O at a content of less than 2000 ppm, advantageously less than 1000 ppm, preferably less than 750 ppm, more preferentially less than 550 ppm, in particular less than 100 ppm of H₂O,
at least one, two, three, four, five, six, seven, eight, nine, ten, eleven, twelve, thirteen, or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   1,2-dichloro-1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloro-2-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,1-chloro-2,2,2-trifluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,1,1-trichloro-2,2-difluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm, and
   1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm;

based on the total weight of the composition.

According to another preferred embodiment, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane,
H₂O at a content of less than 2000 ppm, advantageously less than 1000 ppm, preferably less than 750 ppm, more preferentially less than 550 ppm, in particular less than 100 ppm H₂O, more particularly less than 10 ppm of H₂O,
at least one, two, three, four, five, six, seven, eight, nine, ten, or all of the additional compounds selected from the group consisting of: 1,1,1-trifluoroethane, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 1,2-dichloro-1-fluoroethylene, 1,1-dichloro-2-fluoroethylene, 1-chloro-2-fluoroethane and 1-chloro-1-fluoroethane.

Preferably, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane,
H₂O at a content of less than 2000 ppm, advantageously less than 1000 ppm, preferably less than 750 ppm, more preferentially less than 550 ppm, in particular less than 100 ppm H₂O, more particularly less than 10 ppm of H₂O,
at least one, two, three, four, five, six, seven, eight, nine, ten, or all of the additional compounds selected from the group consisting of:
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm,
   1-chloro-2,2,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm,
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,1-dichloro-2-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1-chloro-2-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm, and
   1-chloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm;
   based on the total weight of the composition.

According to another aspect of the present invention, a composition comprising 1-chloro-2,2-difluoroethane, 1-chloro-2-fluoroethane and 1-chloro-1-fluoroethane is provided. Advantageously, said composition comprises at least 40% by weight, advantageously at least 50% by weight, preferably at least 60% by weight, in particular at least 80% by weight, more particularly at least 90% by weight, favorably at least 95% by weight of 1-chloro-2,2-difluoroethane; a content of 1-chloro-2-fluoroethane of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm; and a content of 1-chloro-1-fluoroethane of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm; based on the total weight of the composition.

According to a preferred embodiment, said composition may also comprise at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1,1-chloro-2,2,2-trifluoroethane, 1,1,1-trichloro-2,2-difluoroethane, 1-chloroethylene, 1-fluoroethylene, H₂O and HCl.

Advantageously, said at least one additional compound is selected from the group consisting of 1,1-difluoroethylene, 1-chloro-1-fluoroethylene, 1,1-difluoroethane, 1,1-dichloroethylene, 1,2-dichloro-1-fluoroethylene, 1,2-dichloro-1,1-difluoroethane, 1,1-dichloro-2,2-difluoroethane, 1,2-dichloro-1-fluoroethylene, 1,1-chloro-2,2,2-trifluoroethane, 1,1,1-trichloro-2,2-difluoroethane, 1-chloroethylene and 1-fluoroethylene.

Thus, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane;
a content of 1-chloro-2-fluoroethane of less than 5% by weight, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm;
a content of 1-chloro-1-fluoroethane of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm;
at least one, two, three, four, five, six, seven, eight, nine, ten, eleven or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   1,2-dichloro-1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,1-chloro-2,2,2-trifluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,1,1-trichloro-2,2-difluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm, and
   1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm;
based on the total weight of the composition.

Advantageously, said at least one additional compound may also be selected from the group consisting of 1,1,1-trifluoroethane, 1,1-difluoroethane, trans-1-chloro-2-fluoroethylene, 1,1,2-trifluoroethane, 1-chloro-1,2,2-trifluoroethane, 1-chloro-2,2,2-trifluoroethane, cis-1-chloro-2-fluoroethylene, 1,2-dichloro-1-fluoroethylene, and 1,1-dichloro-2-fluoroethylene.

Thus, said composition may comprise:
at least 40% by weight, advantageously at least 50% by weight, preferably at least 60% by weight, in particular at least 80% by weight, more particularly at least 90% by weight, favorably at least 95% by weight of 1-chloro-2,2-difluoroethane;
a content of 1-chloro-2-fluoroethane of less than 5% by weight, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm;
a content of 1-chloro-1-fluoroethane of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm;
at least one, two, three, four, five, six, seven, eight, or all of the additional compounds selected from the group consisting of:
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm,
   1-chloro-2,2,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm,
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,1-dichloro-2-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm, based on the total weight of the composition.

Optionally, said composition may comprise H₂O at a content of less than 2000 ppm, advantageously less than 1000 ppm, preferably less than 750 ppm, more preferentially less than 550 ppm, in particular less than 100 ppm based on the total weight of the composition. Optionally, said composition may comprise HCl. The pH of said composition is between 6 and 9, advantageously between 6.5 and 8.5, preferably between 6.5 and 8.0, more preferentially between 7.0 and 8.0.

According to another aspect of the present invention, said composition comprises at least 30% by weight of 1-chloro-2,2-difluoroethane based on the total weight of the composition, and hydrochloric acid.

Preferably, said composition comprises at least 40% by weight of 1-chloro-2,2-difluoroethane, based on the total weight of the composition, advantageously at least 50% by weight of 1-chloro-2,2-difluoroethane, preferably at least 60% by weight of 1-chloro-2,2-difluoroethane, in particular at least 80% by weight of 1-chloro-2,2-difluoroethane, more particularly at least 90% by weight of 1-chloro-2,2-difluoroethane, favorably at least 95% by weight of 1-chloro-2,2-difluoroethane.

Preferably, said composition has a pH of between 6 and 9, preferably between 6.5 and 8.

According to a preferred embodiment, said composition comprises at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O.

Advantageously, said composition comprises less than 40% by weight of said at least one additional compound, based on the total weight of the composition, advantageously less than 35% by weight, preferably less than 30% by weight, more preferentially less than 25% by weight, in particular less than 20% by weight, more particularly less than 10% by weight of said at least one additional compound, based on the total weight of the composition.

Preferably, the composition comprises less than 5% by weight of said at least one additional compound, based on the total weight of the composition, advantageously less than 4% by weight of said at least one additional compound, preferably less than 3% by weight of said at least one additional compound, more preferentially less than 2% by weight of said at least one additional compound, in particular less than 1% by weight of said at least one additional compound, more particularly less than 1000 ppm of said at least one additional compound, based on the total weight of the composition.

In particular, said composition may comprise at least 40% by weight of 1-chloro-2,2-difluoroethane based on the total weight of the composition, advantageously at least 50% by weight of 1-chloro-2,2-difluoroethane, preferably at least 60% by weight of 1-chloro-2,2-difluoroethane, in particular at least 80% by weight of 1-chloro-2,2-difluoroethane, more particularly at least 90% by weight of 1-chloro-2,2-difluoroethane, favorably at least 95% by weight of 1-chloro-2,2-difluoroethane based on the total weight of the composition, and hydrochloric acid, such that the pH of said composition is between 6 and 9, advantageously between 6.5 and 8.5, preferably between 6.5 and 8.0, more preferentially between 7.0 and 8.0; and at least one, two, three four or all of the additional compounds selected from the group consisting of H₂O, trans-1,2-dichloroethylene, 1,1-dichloroethylene, 1,1,2-trifluoroethane and 1-chloro-2,2,2-trifluoroethane. More particularly, said composition may comprise at least 40% by weight of 1-chloro-2,2-difluoroethane based on the total weight of the composition, advantageously at least 50% by weight of 1-chloro-2,2-difluoroethane, preferably at least 60% by weight of 1-chloro-2,2-difluoroethane, in particular at least 80% by weight of 1-chloro-2,2-difluoroethane, more particularly at least 90% by weight of 1-chloro-2,2-difluoroethane, favorably at least 95% by weight of 1-chloro-2,2-difluoroethane based on the total weight of the composition, and hydrochloric acid, such that the pH of said composition is between 6 and 9, advantageously between 6.5 and 8.5, preferably between 6.5 and 8.0, more preferentially between 7.0 and 8.0; and at least one, two, three, four or all of the additional compounds selected from the group consisting of:
H₂O at a content of less than 1% by weight based on the total weight of the composition, advantageously less than 5000 ppm, preferably less than 3000 ppm, more preferentially less than 1000 ppm, in particular less than 750 ppm, more particularly less than 500 ppm, favorably less than 250 ppm, more favorably less than 100 ppm, even more favorably less than 50 ppm, most favorably less than 10 ppm,
trans-1,2-dichloroethylene at a content of less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm,
1,1-dichloroethylene at a content of less than 2% by weight based on the total weight of the composition, advantageously less than 1% by weight, preferably less than 0.5% by weight, more preferentially less than 1000 ppm, in particular less than 500 ppm, more particularly less than 100 ppm, favorably less than 50 ppm, more favorably less than 10 ppm, even more favorably less than 1 ppm,
1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm, and
1-chloro-2,2,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm;
based on the total weight of the composition.

According to another aspect, the present invention provides a composition comprising at least 30% by weight of 1-chloro-2,2-difluoroethane based on the total weight of the composition; said composition being devoid of 1,1-dichloroethylene. According to a preferred embodiment, said composition comprises at least 40% by weight of 1-chloro-2,2-difluoroethane, based on the total weight of the composition, advantageously at least 50% by weight of 1-chloro-2,2-difluoroethane, preferably at least 60% by weight of 1-chloro-2,2-difluoroethane, in particular at least 80% by weight of 1-chloro-2,2-difluoroethane, more particularly at least 90% by weight of 1-chloro-2,2-difluoroethane, favorably at least 95% by weight of 1-chloro-2,2-difluoroethane.

Preferably, said composition may comprise at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl.

According to a preferred embodiment, said composition comprises less than 40% by weight of said at least one additional compound, based on the total weight of the composition, advantageously less than 35% by weight, preferably less than 30% by weight, more preferentially less than 25% by weight, in particular less than 20% by weight, more particularly less than 10% by weight of said at least one additional compound, based on the total weight of the composition. Preferably, the composition comprises less than 5% by weight of said at least one additional compound, based on the total weight of the composition, advantageously less than 4% by weight of said at least one additional compound, preferably less than 3% by weight of said at least one additional compound, more preferentially less than 2% by weight of said at least one additional compound, in particular less than 1% by weight of said at least one additional compound, more particularly less than 1000 ppm of said at least one additional compound, based on the total weight of the composition.

According to a preferred embodiment, said composition, devoid of 1,1-dichloroethylene, comprises 1-chloro-2,2-difluoroethane and at least one of the compounds selected from the group consisting of trans-1,2-dichloroethylene and H₂O. Preferably, said composition, devoid of 1,1-dichloroethylene, comprises:
at least 40% by weight of 1-chloro-2,2-difluoroethane, advantageously at least 50% by weight, preferably at least 60% by weight, in particular at least 80% by weight, more particularly at least 90% by weight, favorably at least 95% by weight,
and at least one or both compounds selected from the group consisting of:
   trans-1,2-dichloroethylene at a content of less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm,
   H₂O at a content of less than 1% by weight based on the total weight of the composition, advantageously less than 5000 ppm, preferably less than 3000 ppm, more preferentially less than 1000 ppm, in particular less than 750 ppm, more particularly less than 500 ppm, favorably less than 250 ppm, more favorably less than 100 ppm, even more favorably less than 50 ppm, most favorably less than 10 ppm, based on the total weight of the composition.

According to a preferred embodiment, said composition, devoid of 1,1-dichloroethylene, comprises 1-chloro-2,2-difluoroethane and at least one of the compounds selected from the group consisting of trans-1,2-dichloroethylene, H₂O, 1-chloro-2,2,2-trifluoroethane and 1,1,2-trifluoroethane.

Preferably, said composition, devoid of 1,1-dichloroethylene, comprises:
at least 40% by weight of 1-chloro-2,2-difluoroethane, advantageously at least 50% by weight, preferably at least 60% by weight, in particular at least 80% by weight, more particularly at least 90% by weight, favorably at least 95% by weight,
and at least one, two three or all of the compounds selected from the group consisting of:
   trans-1,2-dichloroethylene at a content of less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm,
   H₂O at a content of less than 1% by weight based on the total weight of the composition, advantageously less than 5000 ppm, preferably less than 3000 ppm, more preferentially less than 1000 ppm, in particular less than 750 ppm, more particularly less than 500 ppm, favorably less than 250 ppm, more favorably less than 100 ppm, even more favorably less than 50 ppm, most favorably less than 10 ppm,
   1-chloro-2,2,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm, based on the total weight of the composition.

According to a particular embodiment, said composition, devoid of 1,1-dichloroethylene, comprises at least 99.5% by weight of 1-chloro-2,2-difluoroethane, less than 50 ppm of 1,1,2-trifluoroethane, less than 100 ppm of 1-chloro-2,2,2-trifluoroethane and less than 5000 ppm of trans-1,2-dichloroethylene.

According to another particular embodiment, said composition, devoid of 1,1-dichloroethylene, comprises 1-chloro-2,2-difluoroethane, trans-1,2-dichloroethylene, H₂O, 1-chloro-2,2,2-trifluoroethane, 1,1,2-trifluoroethane and at least one of the additional compounds selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-1,2,2-trifluoroethane, cis-1-chloro-2-fluoroethylene, 2,2-dichloro-1,1,1-trifluoroethane, 1,2-dichloro-1,1-difluoroethane, 1,1-dichloro-2,2-difluoroethane, cis-1,2-dichloroethylene, 1,2,2-trichloro-1,1-difluoroethane, 1,2-dichloro-1-fluoroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, and 1,1,2,2-tetrachloroethane.

Preferably, said composition, devoid of 1,1-dichloroethylene, comprises
at least 40% by weight of 1-chloro-2,2-difluoroethane, advantageously at least 50% by weight, preferably at least 60% by weight, in particular at least 80% by weight, more particularly at least 90% by weight, favorably at least 95% by weight,
trans-1,2-dichloroethylene at a content of less than 30% by weight based on the total weight of the composition, advantageously less than 20% by weight, preferably less than 15% by weight, more preferentially less than 10% by weight, in particular less than 5% by weight, more particularly less than 1% by weight, favorably less than 7500 ppm, more favorably less than 5000 ppm,
1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm,
H₂O at a content of less than 1% by weight based on the total weight of the composition, advantageously less than 5000 ppm, preferably less than 3000 ppm, more preferentially less than 1000 ppm, in particular less than 750 ppm, more particularly less than 500 ppm, favorably less than 250 ppm, more favorably less than 100 ppm, even more favorably less than 50 ppm, most favorably less than 10 ppm,
1-chloro-2,2,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm,
at least one, two, three, four, five, six, seven, eight, nine, ten, eleven, twelve, thirteen, fourteen, fifteen, sixteen, seventeen, eighteen, nineteen, twenty, or all of the additional compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   chlorotrifluoroethylene at a content of less than 1000 ppm, advantageously less than 500 ppm, preferably less than 250 ppm, more preferentially less than 100 ppm, in particular less than 50 ppm;
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm.
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   2,2-dichloro-1,1,1-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   1,2,2-trichloro-1,1-difluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm.
   1,2-dichloro-1-fluoroethane at a content of less than 10% by weight based on the total weight of said composition, advantageously less than 9% by weight, preferably less than 8% by weight, more preferentially less than 7% by weight, in particular less than 6% by weight, more particularly less than 5% by weight, favorably less than 4% by weight,
   1,1,2-trichloro-1-fluoroethane at a content of less than 2% by weight based on the total weight of the composition, advantageously less than 1% by weight, preferably less than 5000 ppm, more preferentially less than 2500 ppm, in particular less than 1000 ppm, more particularly less than 500 ppm, favorably less than 100 ppm,
   trichloroethylene at a content of less than 3% by weight based on the total weight of the composition, advantageously less than 2% by weight, preferably less than 10 000 ppm, more preferentially less than 7500 ppm, in particular less than 5000 ppm, more particularly less than 3500 ppm,
   1,1,2-trichloro-2-fluoroethane at a content of less than 5% by weight based on the total weight of the composition, advantageously less than 4% by weight, preferably less than 3% by weight, more preferentially less than 2% by weight, in particular less than 1% by weight,
   1,1,2-trichloroethane at a content of less than 5% by weight based on the total weight of the composition, advantageously less than 4% by weight, preferably less than 3% by weight, more preferentially less than 2% by weight, in particular less than 1% by weight,
   1,1,2,2-tetrachloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   tetrachloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
   1,1,2,2-tetrachloroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm,
based on the total weight of the composition.

According to another particular embodiment, said composition, devoid of 1,1-dichloroethylene, comprises 1-chloro-2,2-difluoroethane and at least one, two, three, four, five, six, seven, eight, nine, ten, eleven, twelve, or all of the compounds selected from the group consisting of 1,1-difluoroethylene, 1-chloro-1-fluoroethylene, 1,1-difluoroethane, 1,2-dichloro-1-fluoroethylene, 1,2-dichloro-1,1-difluoroethane, 1,1-dichloro-2,2-difluoroethane, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and optionally H₂O. Preferably, said composition, devoid of 1,1-dichloroethylene, comprises:
at least 40% by weight of 1-chloro-2,2-difluoroethane, advantageously at least 50% by weight, preferably at least 60% by weight, in particular at least 80% by weight, more particularly at least 90% by weight, favorably at least 95% by weight,
and at least one, two, three, four, five, six, seven, eight, nine, ten, eleven, twelve, or all of the compounds selected from the group consisting of:
   1,1-difluoroethylene at a content of less than 1% by weight based on the total weight of the composition, advantageously less than 0.5% by weight, preferably less than 1000 ppm, more preferentially less than 500 ppm, in particular less than 250 ppm, more particularly less than 200 ppm, favorably less than 50 ppm, more favorably less than 10 ppm, even more favorably less than 1 ppm,
   1-chloro-1-fluoroethylene at a content of less than 1% by weight based on the total weight of the composition, advantageously less than 5000 ppm, preferably less than 2500 ppm, more preferentially less than 1000 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,2-dichloro-1,1-difluoroethane at a content of less than 5% by weight based on the total weight of the composition, advantageously less than 4% by weight, preferably less than 3% by weight, more preferentially less than 2% by weight, in particular less than 1% by weight, more particularly less than 5000 ppm, favorably less than 2500 ppm,
   1,1-dichloro-2,2-difluoroethane at a content of less than 5% by weight based on the total weight of the composition, advantageously less than 4% by weight, preferably less than 3% by weight, more preferentially less than 2% by weight, in particular less than 1% by weight,
   1,2-dichloro-1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,1-chloro-2,2,2-trifluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1,1,1-trichloro-2,2-difluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloro-2-fluoroethane at a content of less than 5% by weight, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm, and
   1-fluoroethylene at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm; and
   optionally H₂O at a content of less than 2000 ppm, advantageously less than 1000 ppm, preferably less than 750 ppm, more preferentially less than 550 ppm, in particular less than 100 ppm, more particularly less than 10 ppm, based on the total composition.

According to another particular embodiment, said composition, devoid of 1,1-dichloroethylene, comprises 1-chloro-2,2-difluoroethane and at least one, two, three, four, five, six, seven, eight, nine, or all of the compounds selected from the group consisting of 1,1,1-trifluoroethane, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 1,2-dichloro-1-fluoroethylene, 1,1-dichloro-2-fluorethylene, 1-chloro-2-fluoroethane and 1-chloro-1-fluoroethane. Preferably, said composition, devoid of 1,1-dichloroethylene, comprises 1-chloro-2,2-difluoroethane, 1-chloro-2,2,2-trifluoroethane and at least one, two, three, four, five, six, seven, eight, nine, or all of the compounds selected from the group consisting of 1,1,1-trifluoroethane, 1,1-difluoroethane, trans-1-chloro-2-fluoroethylene, 1,1,2-trifluoroethane, 1-chloro-1,2,2-trifluoroethane, cis-1-chloro-2-fluoroethylene, 1,1-dichloro-2-fluorethylene, 1,2-dichloro-1-fluoroethylene, 1-chloro-2-fluoroethane and 1-chloro-1-fluoroethane.

In particular, said composition, devoid of 1,1-dichloroethylene, comprises:
at least 40% by weight of 1-chloro-2,2-difluoroethane, advantageously at least 50% by weight, preferably at least 60% by weight, in particular at least 80% by weight, more particularly at least 90% by weight, favorably at least 95% by weight,
1-chloro-2,2,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 60 ppm,
and at least one, two, three, four, five, six, seven, eight, nine, or all of the compounds selected from the group consisting of:
   1,1,1-trifluoroethane at a content of less than 5% by weight based on the total weight of the composition, advantageously less than 4% by weight, preferably less than 2% by weight, more preferentially less than 1% by weight, in particular less than 250 ppm, more particularly less than 200 ppm, favorably less than 50 ppm, more favorably less than 10 ppm, even more favorably less than 1 ppm,
   1,1-difluoroethane at a content of less than 1% by weight based on the total weight of the composition, advantageously less than 5000 ppm, preferably less than 2500 ppm, more preferentially less than 1000 ppm, in particular less than 500 ppm, more particularly less than 300 ppm,
   trans-1-chloro-2-fluoroethylene at a content of less than 2% by weight based on the total weight of the composition, advantageously less than 1% by weight, preferably less than 8000 ppm, more preferentially less than 5000 ppm, in particular less than 4000 ppm, more particularly less than 3000 ppm,
   1,1,2-trifluoroethane at a content of less than 5% by weight based on the total weight of the composition, advantageously less than 4% by weight, preferably less than 3% by weight, more preferentially less than 2% by weight, in particular less than 1% by weight, more particularly less than 1000 ppm, favorably less than 500 ppm, more favorably less than 100 ppm, even more favorably less than 50 ppm,
   1-chloro-1,2,2-trifluoroethane at a content of less than 1% by weight based on the total weight of the composition, advantageously less than 5000 ppm, preferably less than 4000 ppm, more preferentially less than 3000 ppm, in particular less than 2000 ppm, more particularly less than 1600 ppm,
   cis-1-chloro-2-fluoroethylene at a content of less than 5% by weight based on the total weight of the composition, advantageously less than 4% by weight, preferably less than 3% by weight, more preferentially less than 2% by weight, in particular less than 1% by weight, more particularly less than 8000 ppm,
   1,2-dichloro-1-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1,1-dichloro-2-fluoroethylene at a content of less than 1% by weight based on the total weight of the composition, advantageously less than 5000 ppm, preferably less than 4000 ppm, more preferentially less than 3000 ppm, in particular less than 2000 ppm, more particularly less than 1000 ppm,
   1-chloro-2-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   1-chloro-1-fluoroethane at a content of less than 5% by weight based on the total weight of the composition, advantageously less than 2% by weight, preferably less than 1% by weight, more preferentially less than 5000 ppm, in particular less than 1000 ppm, more particularly less than 500 ppm, favorably less than 100 ppm, more favorably less than 50 ppm,
   optionally H₂O at a content of less than 2000 ppm, advantageously less than 1000 ppm, preferably less than 750 ppm, more preferentially less than 550 ppm, in particular less than 100 ppm, more particularly less than 10 ppm based on the total weight of the composition.

Preferably, any one of said compositions devoid of 1,1-dichloroethylene described in the present application may also comprise HCl at a content such that the composition has a pH of between 6 and 9, advantageously between 6.5 and 8.5, preferably between 6.5 and 8.0, more preferentially between 7.0 and 8.0.

According to another aspect of the present invention, the compounds described in the present application may be used as starting product for the synthesis of 2,2-difluoroethylamine.

The compositions according to the present invention are also applicable in a process for preparing a compound of formula CF₂CH₂NHCH₂A (I) comprising the steps of:

(a) providing a composition according to the present invention,
(b) reaction between the composition from step (a) and an amine of formula ACH₂NH₂ (II) in order to obtain the compound of formula CF₂CH₂NHCH₂A (I) in which A is selected from the group consisting of a pyrid-2-yl, pyrid-4-yl or pyrid-3-yl radical, optionally substituted in position 6 by a fluorine, chlorine or bromine atom, or a methyl, trifluoromethyl or trifluoromethoxy radical, or is a pyridazin-3-yl radical optionally substituted in position 6 by a chlorine atom or a methyl radical, or is a pyrazin-3-yl, 2-chloropyrazin-5-yl or 1,3-thiazol-5-yl radical optionally substituted in position 2 by a chlorine atom or a methyl radical, or is a pyrimidinyl, pyrazolyl, thiophenyl, oxazolyl, isoxazolyl, 1,2,4-oxadiazolyl, isothiazolyl, 1,2,4-triazolyl or 1,2,5-thiadiazolyl radical optionally substituted by a fluorine, chlorine or bromine atom or a cyano or nitro group or C₁-C₄-alkyl optionally substituted by F and/or Cl, C₁-C₃-alkylthio optionally substituted by F and/or Cl, C₁-C₃-alkylsulfonyl optionally substituted by F and/or Cl, or is a pyrid-3-yl radical of formula

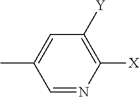

in which X is F, Cl, Br, I, C₁-C₆ alkyl, C₁-C₆-haloalkyl; and Y is F, Cl, Br, I, C₁-C₆ alkyl, C₁-C₆-haloalkyl, C₁-C₆-haloalkoxy, —N₃, —CN.

According to another aspect of the present invention, the compositions described in the present application may be used as reagents for the synthesis of vinylidene fluoride and/or 1,2-difluoroethylene.

According to another aspect of the present invention, the compositions described in the present application may be used as foam blowing agent or as refrigerant.

According to another aspect of the present invention, the compositions described in the present application may be used as reagent or synthesis intermediate for the preparation of 2,2-difluoroethanol or for the preparation of compounds comprising a sulfone, ether, ester or methacrylate functional group or for the preparation of ethyl difluoro bromoacetate.

The compositions according to the present invention may be obtained by different processes. The compounds present in said composition may vary as a function of the process used. For example, a composition according to the present invention comprising 1-chloro-2,2-difluoroethane may be obtained from 1,1,2-trichloroethane or 1-chloro-2,2,2-trifluoroethane or 1,1-difluoroethane by suitable processes.

Said compositions may be obtained by mixing the different constituents thereof in the desired proportions. For example, 1-chloro-2,2-difluoroethane is commercially available (Synquest Laboratories, product: 1100-7-09), and trans-1,2-dichloroethylene, cis-1,2-dichloroethylene, 1,1-dichloroethylene and 1,1,2-trifluoroethane are available from Sigma-Aldrich (D62209, D62004, 163023, 270369).

Alternatively, said compositions may be obtained by chemical synthesis from commercial precursors.

### EXAMPLES

Compositions 1 to 5 detailed in table 1 were prepared. Unless otherwise indicated, the contents are expressed by weight relative to the total weight of the composition. Compositions 1 to 5 have a pH of between 6 and 9. The compositions were analyzed by gas chromatography-mass spectrometry (GC-MS) in electron ionization mode (70 eV) and quadripolar analyzer. A calibration curve was produced for reference compounds at different concentrations (10%, 5%, 1%, 500 ppm, 100 ppm). A calibration curve was generated for 1,1,1-trifluoroethane, 1,1-difluoroethane, chlorotrifluoroethylene, 1-chloro-2,2,2-trifluoroethane, cis-1-chloro-2-fluoroethylene, 1-chloro-2,2-difluoroethane, chloropropene, 1,2-dichloro-1,1-difluoroethane, trans-1,2-dichloroethylene, 1,2,2-trichloro-1,1-difluoroethane, trichloroethylene, 1,1,2-trichloroethane.

The chromatographic conditions used are as follows:
Column: ZB-5 50 m*0.32 mm*5 μm
Temperature program: 40° C. (10 min)->4° C./min->250° C.
Split/splitless injector: 200° C.
Flow rate: 1 ml/min
Apparatus: 10197125—GC/MS Shimadzu

The contents of H₂O and of HCl are determined in accordance with the procedure described in AHRI Standard 700-201, respectively at point 5.4 and 5.7 and Appendix C to AHRI Standard 700-2012, respectively Part 2 and Part 1.

TABLE 1

| Composition | 1 | 2 | 3 | 4 | 5 |
|---|---|---|---|---|---|
| F142 | 98 | 98 | 99.9 | 49.1 | 64.0 |
| F1130trans | 2 | 2 | 49 ppm | 10.7 | 5.8 |
| H₂O | <1 ppm | 10 ppm | <1 ppm | 500 ppm | <1 ppm |
| F143 | | | <1 ppm | 0.5 | 1.9 |
| F1130a | <1 ppm | 50 ppm | <1 ppm | 0.3 | <1 ppm |
| F1130cis | | | | 27.7 | 15.6 |
| F133a | | | <1 ppm | 2.9 | 0.6 |
| F141 | | | | 3.6 | 3.2 |
| F132a | | | | 1.3 | 1.6 |
| F132b | | | | 0.8 | 0.2 |
| F1131cis | | | | 1.1 | 0.7 |
| F143a | | | | | 1.1 |
| F131 | | | | | 2.1 |
| F140 | | | | | 1.2 |
| F130 | | | | | 1.3 |
| Others | | | | 1.9 | 0.7 |

1. A composition comprising 1-chloro-2,2-difluoroethane and at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,1-dichloroethylene, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; said composition comprising at least 30% by weight of 1-chloro-2,2-difluoroethane, based on the total weight of the composition.

2. The composition as claimed in claim 1, comprising 1-chloro-2,2-difluoroethane and at least one additional compound selected from the group consisting of 1,1-difluoroethylene, chlorotrifluoroethylene, 1-chloro-1-fluoroethylene, 1,1-difluoroethane, trans-1-chloro-2-fluoroethylene, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,1,1-trichloroethane, 1,1-dichloroethane, 1,1,2-trichloro-1-fluoroethane, trichloroethylene, 1,1,2-trichloro-2-fluoroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene, H₂O and HCl; said composition comprising at least 30% by weight of 1-chloro-2,2-difluoroethane, based on the total weight of the composition.

3. The composition as claimed in claim 1, characterized in that it wherein the composition comprises at least 40% by weight of 1-chloro-2,2-difluoroethane, based on the total weight of the composition.

4. The composition as claimed in claim 1, wherein the composition comprises less than 5% by weight of said at least one additional compound, based on the total weight of the composition.

5. The composition as claimed in claim 1, wherein the composition further comprises one of the compounds selected from the group consisting of trans-1,2-dichloroethylene, 1,1,2-trifluoroethane and 1,2-dichloro-1-fluoroethane.

6. The composition as claimed in claim 1, wherein the composition comprises 1-chloro-2,2-difluoroethane, 1,1-dichloroethylene and optionally or not at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1,2-dichloro-1-fluoroethylene, 1,1-dichloro-2-fluoroethylene, 1,2-dichloro-1,1-difluoroethane, trans-1,2-dichloroethylene, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O.

7. The composition as claimed in claim 1, wherein the composition comprises 1-chloro-2,2-difluoroethane, 1,1-dichloroethylene, trans-1,2-dichloroethylene and optionally at least one additional compound selected from the group consisting of 1,1-difluoroethylene, 1,1,1-trifluoroethane, chlorotrifluoroethylene, 1-chloro-1-fluoroethylene, 1,1-difluoroethane, trans-1-chloro-2-fluoroethylene, 1,1,2-trifluoroethane, 1-chloro-2,2,2-trifluoroethane, 1-chloro-1,2,2-trifluoroethane, cis-1-chloro-2-fluoroethylene, 2,2-dichloro-1,1,1-trifluoroethane, 1,1-dichloro-1-fluoroethane, 1-chloro-2,2-difluoroethane, 1,2-dichloro-1-fluoroethylene, 1,1-dichloro-2-fluoroethylene, 1,2-dichloro-1,1-difluoroethane, 1,1-dichloro-2,2-difluoroethane, cis-1,2-dichloroethylene, 1,2,2-trichloro-1,1-difluoroethane, 1,2-dichloro-1-fluoroethane, 1,1,1-trichloroethane, 1,1-dichloroethane, 1,1,2-trichloro-1-fluoroethane, trichloroethylene, 1,1,2-trichloro-2-fluoroethane, 1,1,2-trichloroethane, 1,1,2,2-tetrachloro-1-fluoroethane, tetrachloroethylene, 1,1,1,2-tetrachloroethane, 1,1,2,2-tetrachloroethane, 1,1,1,2,2-pentachloroethane, tetrachloromethane, 1,2,2-trichloro-1-fluoroethylene, isomers of chloropropene, 1,2-dichloro-1-fluoroethylene, 1-chloro-2-fluoroethane, 1-chloro-1-fluoroethane, 1,1-chloro-2,2,2-trifluoroethane, 1,1,1-trichloro-2,2-difluoroethane, 1-chloroethylene, 1-fluoroethylene and H₂O.

8. The composition as claimed in claim 1, wherein the composition comprises 1-chloro-2,2-difluoroethane, 1,1-dichloroethylene, trans-1,2-dichloroethylene, 1-chloro-2,2,2-trifluoroethane and 1,1,2-trifluoroethane.

9. The composition as claimed in claim 8, wherein the composition comprises at least 99.5% by weight of 1-chloro-2,2-difluoroethane, less than 50 ppm of 1,1,2-trifluoroethane, less than 100 ppm of 1-chloro-2,2,2-trifluoroethane and less than 5000 ppm of trans-1,2-dichloroethylene and less than 50 ppm of 1,1-dichloroethylene.

10. The composition as claimed in claim 9, wherein the composition comprises less than 500 ppm of H₂O.

11. A method of synthesis of 2,2-difluoroethylamine, the method comprising using a composition as claimed in claim 1 as starting product for the synthesis of 2,2-difluoroethylamine.

12. A process for preparing a compound of formula CF₂CH₂NHCH₂A (I), comprising the steps of:
(a) providing a composition as claimed in claim 1;
(b) reaction between the composition from step (a) and an amine of formula ACH₂NH₂ (II) in order to obtain the compound of formula CF₂CH₂NHCH₂A (I) in which A is selected from the group consisting of a pyrid-2-yl, pyrid-4-yl or pyrid-3-yl radical, optionally substituted in position 6 by a fluorine, chlorine or bromine atom, or a methyl, trifluoromethyl or trifluoromethoxy radical, or is a pyridazin-3-yl radical optionally substituted in position 6 by a chlorine atom or a methyl radical, or is a pyrazin-3-yl, 2-chloropyrazin-5-yl or 1,3-thiazol-5-yl radical optionally substituted in position 2 by a chlorine atom or a methyl radical, or is a pyrimidinyl, pyrazolyl, thiophenyl, oxazolyl, isoxazolyl, 1,2,4-oxadiazolyl, isothiazolyl, 1,2,4-triazolyl or 1,2,5-thiadiazolyl radical optionally substituted by a fluorine, chlorine or bromine atom or a cyano or nitro group or C₁-C₄-alkyl optionally substituted by F and/or Cl, C₁-C₃-alkylthio optionally substituted by F and/or Cl, C₁-C₃-alkylsulfonyl optionally substituted by F and/or Cl, or is a pyrid-3-yl radical of formula 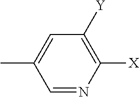
in which X is F, Cl, Br, I, C₁-C₆ alkyl, C₁-C₆-haloalkyl; and Y is F, Cl, Br, I, C₁-C₆ alkyl, C₁-C₆-haloalkyl, C₁-C₆-haloalkoxy, —N₃, —CN.

13. A method of synthesis of vinylidene fluoride and/or 1,2-difluoroethylene, the method comprising using the composition as claimed in claim 1 as reagents for the synthesis of vinylidene fluoride and/or 1,2-difluoroethylene.

14. A foam blowing agent or a refrigerant comprising the composition as claimed in claim 1.

15. A method comprising using the composition as claimed in claim 1 as reagent or synthesis intermediate for the preparation of 2,2-difluoroethanol or for the preparation of compounds comprising a sulfone, ether, ester or methacrylate functional group or for the preparation of ethyl difluoro bromoacetate.
